(19) United States
(12) Patent Application Publication
(10) Pub. No.: US 2024/0384316 A1
(43) Pub. Date: Nov. 21, 2024

(54) Purification of Burkholderia Capsular Polysaccharides

(71) Applicant: Board of Regents of the Nevada System of Higher Education, on behalf of the University of Nevada, Reno, NV (US)

(72) Inventors: Paul J. Brett, Reno, NV (US); Mary Nicole Burtnick, Reno, NV (US)

(21) Appl. No.: 18/287,571

(22) PCT Filed: Apr. 29, 2022
(86) PCT No.: PCT/US2022/026994

§ 371 Date: Oct. 19, 2023

Related U.S. Application Data
(60) Provisional application No. 63/182,356, filed on Apr. 30, 2021.

(51) Int. Cl. C12P 19/04; A61K 39/02; C08B 37/00; C12N 1/20

(52) U.S. Cl. CPC C12P 19/04; A61K 39/0208; C08B 37/00; C12N 1/20

(57) ABSTRACT

In one aspect, the present disclosure provides to a method for isolating and purifying a capsular polysaccharide (CPS) from a Burkholderia species. In one embodiment, the Burkholderia species is the O-polysaccharide mutant strain B. thailandensis BT2683. In one embodiment, the CPS is a 6-deoxy-heptan CPS. In another aspect, the present disclosure further provides a Burkholderia CPS isolated and purified by the disclosed method.

### CROSS-REFERENCE TO RELATED APPLICATION

The present application is entitled to priority under 35 U.S.C. § 119(e) to U.S. Provisional Patent Application No. 63/182,356, filed Apr. 30, 2021, which is hereby incorporated by reference in its entirety herein.

### STATEMENT REGARDING FEDERALLY SPONSORED RESEARCH

This invention was made with government support under HDTRA1-18-C-0062 awarded by the Defense Threat Reduction Agency. The government has certain rights in the invention.

### BACKGROUND

Melioidosis is an emerging infectious disease that is being increasingly recognized in tropical regions around the world. While it is known to be endemic in at least 48 different countries in Southeast Asia, South Asia, the Middle East, Africa, Central America and South America, current models predict that the disease is probably endemic in 34 additional countries where it has yet to be reported. Under-recognition of melioidosis is due, in part, to the fact that most cases occur in resource-poor countries with large rural populations and limited microbiological laboratory capabilities. Since the clinical presentations of melioidosis are diverse, ranging from skin abscesses to acute pneumonias and septicemias, diagnosis can be difficult. In 2015, the estimated total global burden of human melioidosis was ˜165,000 cases with ˜89,000 deaths which is equivalent to that of measles and exceeds the levels of leptospirosis and dengue infection, underscoring the potential impact of the disease worldwide.

Burkholderia pseudomallei, the etiologic agent of melioidosis, is a facultative intracellular Gram-negative bacterium that can be isolated from environmental niches such as rice paddies, still or stagnant waters, and moist soils in endemic areas. Humans can acquire B. pseudomallei infections through percutaneous inoculation via skin abrasions during occupational or recreational exposure, inhalation of bacteria in aerosolized dust or water, or ingestion of contaminated water. Most natural infections occur in individuals with one or more risk factors such as diabetes, alcoholism, chronic pulmonary disease, chronic renal disease or thalassemia.

At present, the association between route of infection and the clinical manifestations of melioidosis is not clearly defined. Recent studies, however, have demonstrated a link between inhalation of aerosolized B. pseudomallei during severe weather events and pneumonia. Notably, over half of all melioidosis cases present as pneumonias which can range from mild to severe disease.

In addition to being an important public health concern, B. pseudomallei is also considered a potential biological weapon and is currently categorized as a Tier 1 select agent by U.S. Centers for Disease Control and Prevention. In the event of an intentional release, it is believed that the most likely mode of dissemination would be via infectious aerosols leading to respiratory disease. Since B. pseudomallei is intrinsically resistant to many conventionally used antibiotics, treatment of melioidosis can be complicated. For culture confirmed cases, the currently recommended antibiotic regimens are lengthy and typically involve a minimum of two weeks of intravenous therapy followed by up to six months of oral therapy. The ability of B. pseudomallei to persist inside of host cells makes eradication of infections difficult and even with appropriate chemotherapeutic intervention, relapse is possible. Furthermore, re-infection with a different B. pseudomallei strain can occur following successful treatment.

At present, there are no human vaccines available for immunization against melioidosis. Because of these challenges, the development of medical countermeasures to combat melioidosis has become a priority in recent years. However, the procedures developed for extracting antigens from Burkholderia species are lengthy, expensive, and difficult to scale up, making it difficult to extract the amount of antigen needed for a vaccine. In order to mass produce a vaccine to combat melioidosis or glanders, an infectious disease caused by the related bacterium Burkholderia mallei, a rapid process for extracting large amounts of antigen from B. pseudomallei, B. mallei, or related Burkholderia species is needed.

There is a need in the art for a rapid and cost-effective process for extracting large amounts of polysaccharides from Burkholderia species. The present invention satisfies these unmet needs.

### SUMMARY OF THE INVENTION

The present invention is based on the discovery of a novel method of isolating and purifying Burkholderia capsular polysaccharides. The method provides a high yield of uniform polysaccharide material.

In one aspect, a method is provided for isolating and purifying a capsular polysaccharide (CPS) from a Burkholderia species, the method comprising (a) culturing Burkholderia cells; (b) pelleting the cells, resuspending the cells in water, adding an alcohol to the resuspended cells to form a cell slurry; (c) pelleting the cell slurry, removing the supernatant; (d) adding an alcohol to the supernatant, forming a precipitate comprising the CPS; (e) pelleting the CPS precipitate, resuspending the precipitate in water, dialyzing the resuspended CPS precipitate; (f) concentrating the dialyzed CPS precipitate, solubilizing the CPS in a weak acid, separating the solubilized CPS from a solid impurity; (g) concentrating the solubilized CPS, adding a buffer to the concentrated solubilized CPS to form a solution, loading the solution onto a gel filtration resin; and (h) eluting purified CPS from the gel filtration resin. In some embodiments, the Burkholderia species is Burkholderia thailandensis. In some embodiments, the Burkholderia species is an O-polysaccharide mutant strain of B. thailandensis. In some embodiments, the O-polysaccharide mutant strain of B. thailandensis is B. thailandensisBT2683. In some embodiments, the CPS is a 6-deoxy-heptan CPS.

In some embodiments, step (b) of the method for isolating and purifying a capsular polysaccharide from a Burkholderia species comprises adding an equal volume of ethanol compared to the volume of resuspended cells, with stirring, to the resuspended cells to form a cell slurry.

In some embodiments, step (d) of the method comprises adding ethanol to the supernatant to achieve a final concentration of about 90% (v/v) ethanol, forming a precipitate comprising the CPS.

In some embodiments, step (e) of the method comprises dialyzing the resuspended CPS precipitate against water.

In some embodiments, step (f) of the method comprises solubilizing the CPS in an aqueous solution comprising between about 1% to 3% by volume of acetic acid to form a mixture having a concentration of between about 2 to 7 mg/ml CPS. In some embodiments, the mixture is incubated for about 2 hours at a temperature of about 100° C.

In some embodiments step (g) of the method comprises adding phosphate-buffered saline to the concentrated CPS to form a solution having a concentration of between about 20 to 30 mg/ml of the CPS and loading the solution onto a gel filtration resin, wherein the gel filtration resin comprises a cross-linked dextran gel filtration resin. In some embodiments, step (g) comprises adding deionized water to the concentrated CPS to form a solution having a concentration of between about 20 to 30 mg/ml of the CPS and loading the solution onto a gel filtration resin, wherein the gel filtration resin comprises a cross-linked dextran gel filtration resin.

In some embodiments, step (h) of the method comprises eluting purified CPS from the gel filtration resin using phosphate-buffered saline. In some embodiments, step (h) of the method comprises eluting purified CPS from the gel filtration resin using deionized water. In some embodiments, step (h) of the method is followed by step (i) comprising dialyzing the purified CPS. In some embodiments, the purified CPS is dialyzed against water.

In some embodiments, step (h) or step (i) of the method is followed by step (j) comprising concentrating the purified CPS.

In some embodiments, the alcohol is ethanol.

In one aspect the disclosure provides a Burkholderia CPS isolated and purified by any of the above methods. In some embodiments the Burkholderia CPS is isolated and purified from O-polysaccharide mutant strain B. thailandensis BT2683 by any of the above methods. In some embodiments, a 6-deoxy-heptan CPS is isolated and purified by any of the above methods.

### BRIEF DESCRIPTION OF THE DRAWINGS

The following detailed description of exemplary embodiments of the invention will be better understood when read in conjunction with the appended drawings. For the purpose of illustrating the invention, non-limiting embodiments are shown in the drawings. It should be understood, however, that the invention is not limited to the precise arrangements and instrumentalities of the embodiments shown in the drawings.

FIG. 1 is an image of an analysis of the B. thailandensis capsular polysaccharide (CPS) extract using InBios AMD-LFIs. A 1 μl aliquot of the precipitated CPS re-solubilized in 30 ml of dH₂O was serially diluted to 1/1,000,000 in phosphate-buffered saline (PBS). 50 μl of the diluted sample was then added to an AMD LFI cassette and followed by 3-4 drops of chase buffer.

FIG. 2 is a graph of an analysis of the Sephadex G-50 column fractions using the phenol sulfuric acid assay. 25 μl of each column fraction was mixed with 100 μl dH₂0, 200 μl 5% phenol and 1 ml sulfuric acid. Fractions were read at OD 490 nm on an Eppendorf spectrophotometer.

FIG. 3 is an image of an analysis of the B. thailandensis CPS dialysate using InBios AMD-LFI Cassettes. A 1 μl aliquot of the dialysate was serially diluted in PBS to 1/1,000,000. 50 μl of the diluted sample was then added to an AMD LFI cassette and followed by 3-4 drops of chase buffer.

FIG. 4A-4B depict the ¹H NMR spectra of B. thailandensis CPS samples eluted from Sephadex G-50 columns in PBS (S1; FIG. 4A) or water (S2; FIG. 4B).

FIG. 5A-5B depict size exclusion chromatography (SEC) analysis of G-50 purified Bt-CPS samples. Bt-CPS samples were purified using the phenol extraction method (P-CPS) or the ethanol extraction method (E-CPS), solubilized at 2 mg/ml in PBS and run on a Superdex 200 Increase 10/300 GL column at the same flow rates (P-CPS, FIG. 5A; E-CPS, FIG. 5B).

### DETAILED DESCRIPTION OF THE DISCLOSURE

In one aspect, the present disclosure relates to a novel method of isolating and purifying Burkholderia capsular polysaccharides. In one embodiment, the method comprises isolating and purifying a 6-deoxy-heptan CPS. In one embodiment, the method comprises isolating and purifying a 6-deoxy-heptan CPS from B. thailandensis. In one embodiment, the B. thailandensis strain is genetically engineered to simplify CPS purification. In one embodiment, an E555 strain of B. thailandensis is genetically engineered to simplify CPS purification. In some embodiments, the genetically engineered B. thailandensis strain no longer produces O-polysaccharide. One advantage of the disclosed method is that it takes fewer days than previously disclosed methods of purifying CPS. Another advantage is that disclosed method is more cost effective than previously disclosed methods of purifying CPS. Yet another advantage of the disclosed method is that it is more readily scaled up than previously disclosed methods of purifying CPS. Yet another advantage of the disclosed method is that the use of phenol, ultracentrifugation, and enzymes are avoided during the purification of CPS. Therefore, in some embodiments, the disclosed method can be used to isolate and purify large amounts of 6-deoxy-heptan CPS from B. thailandensis. In one embodiment, the disclosed method can be used to isolate and purify 6-deoxy-heptan CPS that can be used in a vaccine for melioidosis and/or glanders.

In another aspect, the present disclosure relates to a Burkholderia CPS that is isolated and purified using the disclosed method. In one embodiment, the Burkholderia CPS is isolated and purified from O-polysaccharide mutant strain B. thailandensis BT2683 using the disclosed method. In one embodiment, BT2683 is a rmID knockout and thus does not produce O-polysaccharide. In one embodiment, a 6-deoxy-heptan CPS is isolated and purified using the disclosed method.

### Definitions

Unless defined otherwise, all technical and scientific terms used herein have the same meaning as commonly understood by one of ordinary skill in the art to which the invention pertains. Although any methods and materials similar or equivalent to those described herein may be used in the practice for testing of the present invention, the preferred materials and methods are described herein. In describing and claiming the present invention, the following terminology will be used.

It is also to be understood that the terminology used herein is for the purpose of describing particular embodiments only, and is not intended to be limiting.

As used herein, the articles “a” and “an” are used to refer to one or to more than one (i.e., to at least one) of the grammatical object of the article. By way of example, “an element” means one element or more than one element.

As used herein when referring to a measurable value such as an amount, a temporal duration, and the like, the term “about” is meant to encompass variations of ±20% or ±10%, more preferably ±5%, even more preferably ±1%, and still more preferably ±0.1% from the specified value, as such variations are appropriate to perform the disclosed methods.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

As used herein, gel filtration chromatography or size exclusion chromatography (SEC) are used interchangeably, and refer to a chromatographic method in which molecules in aqueous solution are separated based on their differences in size or molecular weight. Gel filtration chromatography is a widely used polymer characterization method because of its ability to provide a good separation and molar mass distribution (Mw) of polymers. Without being limited thereto, the chromatography column is packed with resin material which contain porous beads. Separation of the polymers polysaccharides is achieved by the differential exclusion of the polymers from the pores of the packing material as the sample passes through the bed of porous particles. Without being limited thereto, resin materials can be composed of dextran polymers (Sephadex), agarose (Sepharose), or polyacrylamide (Sephacryl or BioGel P). Exemplary resin materials are Sephadex® G-50, and Superdex 200 Increase 10/300 GL. In some embodiments, the polymers separated by chromatography are polysaccharides. In some embodiments, the polysaccharides separated by gel filtration chromatography are extracted from Burkholderia.

### Methods

In one aspect, the present invention relates to a method for isolating and purifying a capsular polysaccharide (CPS) from a Burkholderia species, the method comprising
(a) culturing Burkholderia cells;
(b) pelleting the cells, resuspending the cells in water, adding an alcohol to the resuspended cells to form a cell slurry;
(c) pelleting the cell slurry, removing the supernatant;
(d) adding an alcohol to the supernatant, forming a precipitate comprising the CPS;
(e) pelleting the CPS precipitate, resuspending the precipitate in water, dialyzing the resuspended CPS precipitate;
(f) concentrating the dialyzed CPS precipitate, solubilizing the CPS in a weak acid, separating the solubilized CPS from a solid impurity;
(g) concentrating the solubilized CPS, adding a buffer to the concentrated CPS to form a solution, loading the solution onto a gel filtration resin; and
(h) eluting purified CPS from the gel filtration resin.

The Burkholderia cells in step (a) can comprise cells of any Burkholderia species known to produce a CPS. Exemplary Burkholderia species include, but are not limited to, Burkholderia pseudomallei, Burkholderia mallei, Burkholderia thailandensis, Burkholderia caryophylli, Burkholderia gladioli, Burkholderia solanacearum, Burkholderia pickettii, Burkholderia cepacia, Burkholderia cenocepacia, and Burkholderia stabilis. In one embodiment, the Burkholderia cells are from the B. pseudomallei species. In another embodiment, the Burkholderia cells are from the B. mallei species. In yet another embodiment, the Burkholderia cells are from the B. thailandensis species. In one embodiment, the Burkholderia cells are the O-polysaccharide mutant strain of B. thailandensis BT2683 (Δrm1D)). In other embodiments, the Burkholderia cells are an O-polysaccharide mutant strain of one of the Burkholderia species described elsewhere herein. Although not wishing to be limited by theory, it is believed that the O-polysaccharide mutant strains eliminate contamination with other polysaccharides (i.e. O-polysaccharide). Additionally, while not wishing to be limited by theory, it is believed that O-polysaccharide deficient mutants in of the Burkholderia species described herein would also simplify purification of the CPS. In one embodiment, the CPS is a 6-deoxy-heptan capsular polysaccharide.

The Burkholderia cells in step (a) can be cultured using standard cell culture methods known to a person of skill in the art. In one embodiment, about 25 ml of M9TG media comprising M9 Minimal Salts and Tryptone is inoculated with a Burkholderia stock. In one embodiment, the inoculated media is incubated at about 37° C. for about 18 hours with vigorous shaking to form a starter culture. In some embodiments, after about 18 hours, a portion of the starter culture is added to multiple flasks containing about 600 ml of M9TG media. The flasks are incubated at about 37° C. for about 24 hours with vigorous shaking.

Once the cell cultures of step (a) have reached sufficient density, the Burkholderia cells are pelleted in step (b). In some embodiments, the cells are pelleted using centrifugation. In one embodiment, the cells are pelleted by centrifugation for about 10 minutes at about 8,000×g. In some embodiments, the pelleted cells are resuspended in about 200 ml of water. In one embodiment, the water is deionized water. In some embodiments, the step of adding an alcohol to the resuspended cells in (b) is performed with stirring. In one embodiment, about an equal volume of alcohol compared the volume of resuspended cells is added, with stirring, to the cells. In some embodiments, the alcohol is ethanol. In some embodiments, the cell slurry of (b) is stirred at room temperature for about 30 minutes.

The cell slurry of (b) is pelleted in step (c). In some embodiments, the cell slurry is pelleted using centrifugation. In one embodiment, the cell slurry is pelleted by centrifugation for about 10 minutes at about 8,000×g. In some embodiments, the step of removing the supernatant in (c) further comprises filtering the supernatant using a 0.45 μm filter.

In step (d), alcohol is added to the supernatant of (c). In one embodiment, the alcohol is ethanol. In some embodiments, the alcohol is added to the supernatant in an amount sufficient to form a precipitate. In some embodiments, wherein the method is used to isolate and purify a 6-deoxy-heptan capsular polysaccharide, the alcohol is added to achieve a final concentration of about 90% (v/v). In one embodiment, the step of adding an alcohol to the supernatant further comprises periodically swirling the alcohol/supernatant mixture at room temperature for about 60 minutes to form a precipitate.

In step (e), the precipitate formed in step (d) is pelleted. In some embodiments, the precipitate is pelleted using centrifugation. In one embodiment, the precipitate is pelleted by centrifugation for about 30 minutes at about 12,000×g. In some embodiments, the pelleted precipitate is resuspended in deionized water. In one embodiment, the resuspended CPS precipitate is dialyzed against water. In some embodiments, the water is deionized water. In one embodiment, the molecular weight cutoff of the dialysis tubing is selected based on the molecular weight of the CPS that is being isolated and purified. In one embodiment wherein the CPS is a 6-deoxy-heptan capsular polysaccharide, the dialysis tubing has a molecular weight cutoff of about 3,500 Daltons.

In step (f), the dialyzed CPS precipitate can be concentrated using any method known to a person of skill in the art. In one embodiment, the CPS precipitate is concentrated by lyophilization. In some embodiments, before the CPS precipitate is concentrated, the dialyzed CPS precipitate is filtered using a 0.45 μm filter. The concentrated CPS is then solubilized in a weak acid. Exemplary weak acids include, but are not limited to, acetic acid, lactic acid, formic acid, citric acid, oxalic acid, uric acid, malic acid, tartaric acid, and combinations thereof. In one embodiment, the weak acid is acetic acid. In one embodiment, the weak acid is an aqueous solution comprising between about 0.1% to 20%, about 0.1% to 15%, about 0.1% to 10%, about 0.1% to 5%, or about 1% to 3% by volume of a weak acid. In some embodiments, the weak acid is an aqueous solution comprising about 2% by volume acetic acid. In one embodiment, the concentrated CPS is solubilized in the weak acid to form a mixture having a concentration of about 0.1 to 30 mg/ml, about 0.1 to 25 mg/ml, about 0.1 to 20 mg/ml, about 0.1 to 15 mg/ml, about 0.1 to 10 mg/ml, about 1 to 10 mg/ml, or about 2 to 7 mg/ml CPS. In some embodiments, the solubilized CPS is incubated for about 2 hours at temperature of about 100° C. The step of separating the solubilized CPS from a solid impurity in (f) can use any method known to a person of skill in the art. In one embodiment, the solubilized CPS is separated from the solid impurity using centrifugation. In one embodiment, the solubilized CPS comprising the solid impurity is centrifuged for about 20 minutes at 17,000×g to pellet the solid impurity. In one embodiment, the supernatant comprising the solubilized CPS is carried on to step (g).

In step (g), the solubilized CPS can be concentrated using any method known to a person of skill in the art. In one embodiment, the solubilized CPS is concentrated by lyophilization. The step of adding a buffer to the concentrated CPS in (g) can use any buffer known or believed to be used for gel filtration (e.g. known or believed to lead to a good separation based on the gel filtration resin to be used and/or the CPS to be purified). In one embodiment, the buffer is phosphate-buffered saline (PBS). In another embodiment, the buffer is water. When the buffer is water, the water may come from any clean water source, such as filtered water, distilled water, or deionized water. In yet another embodiment, the buffer is any biological buffer that does not comprise an amine. In one embodiment, the buffer has a pH of about 7.2. In some embodiments, the buffer is added to the concentrated CPS to form a solution comprising between about 5 to 50 mg/ml, about 5 to 40 mg/ml, about 5 to 30 mg/ml, about 15 to 30 mg/ml, about 20 to 30 mg/ml of the CPS. In some embodiments, the solution of buffer and concentrated CPS is filtered before loading onto a gel filtration resin. In one embodiment, the filter is a 0.45 μm filter. The gel filtration resin can be any chromatography resin known or believed to be useful in purifying capsular polysaccharides. In one embodiment, the gel filtration resin is a resin used for size exclusion chromatography. Exemplary size exclusion gel filtration resins include, but are not limited to, Sephadex®, Sephacryl®, Superdex®, Sepharose®, Toyopearl®, and Bio-Gel® resins. In one embodiment, the gel filtration resin is a cross-linked dextran gel. In some embodiments, the cross-linked dextran gel is Sephadex®. In one embodiment, the gel filtration resin is Sephadex® G-50. In another embodiment, the gel filtration resin is a resin that has similar resolving capabilities to Sephadex® G-50. In some embodiments, the gel filtration resin is a Sephacryl®, Superdex®, Sepharose®, Toyopearl®, or Bio-Gel® resin that has similar resolving capabilities to Sephadex® G-50. In some embodiments, the gel filtration resin has similar molecular weight ranges as the Sephadex® G-50 resin. In one embodiment, the gel filtration resin is equilibrated with the same buffer that is added to the concentrated CPS. In one embodiment, the gel filtration resin is equilibrated with a PBS buffer having a pH of 7.2. In another embodiment, the gel filtration resin is equilibrated with water. In yet another embodiment, the gel filtration resin is equilibrated with a biological buffer that does not comprise an amine.

In step (h), the purified CPS is eluted using any buffer known or believed to be useful in eluting a CPS from a gel filtration resin. In one embodiment, the gel filtration resin is in a standard chromatography column. In one embodiment, the buffer is the same buffer that is added to the concentrated CPS in step (g). Therefore, in one embodiment, the buffer is PBS with a pH of 7.2. In another embodiment, the purified CPS is eluted with water. In yet another embodiment, the purified CPS is eluted with a biological buffer that does not comprise an amine. In one embodiment, a phenol-sulfuric acid assay is used to determine which fractions eluted from the gel filtration resin contain CPS.

In some embodiments, step (h) is followed by step (i) comprising dialyzing the purified CPS. In one embodiment, the purified CPS is dialyzed against water. In some embodiments, the water is deionized water. In one embodiment, the molecular weight cutoff of the dialysis tubing is selected based on the molecular weight of the purified CPS. In one embodiment wherein the purified CPS is a 6-deoxy-heptan capsular polysaccharide, the dialysis tubing has a molecular weight cutoff of about 3,500 Daltons. In some embodiments, the purified CPS is filtered following dialysis. In one embodiment, the filter is a 0.45 μm filter. In one embodiment wherein the purified CPS in step (h) is eluted using water, step (i) is not performed. In one embodiment wherein the purified CPS in step (h) is eluted using PBS, step (i) is performed.

In some embodiments, step (h) or step (i) is followed by step (j) comprising concentrating the purified CPS. The purified CPS can be concentrated using any method known to a person of skill in the art. In one embodiment, the purified CPS is concentrated by lyophilization.

In some embodiments, the method further comprises the steps of (j) adding a buffer to the concentrated CPS to form a solution; (k) loading the solution onto a second gel filtration resin; and (l) eluting purified CPS from the second gel filtration resin. In some embodiments, the second gel filtration resin is selected from the group consisting of Sephadex® G-50, Sephacryl®, Superdex®, Sepharose®, Toyopearl®, or Bio-Gel® resin. In some embodiments, the second gel filtration resin is Superdex 200 Increase 10/300 GL resin.

In one embodiment, the method of isolating and purifying the CPS takes about seven to nine days. In one embodiment, when a CPS buffer is used in steps (g) and (h), the method of isolating and purifying the CPS takes nine days. In another embodiment, when water is used as the buffer in steps (g) and (h), the method of isolating and purifying the CPS takes seven days. Therefore, the disclosed method is faster than previous methods of isolating and purifying CPS which take about twenty days. Further, the disclosed method produces a higher yield of CPS than previous methods. The disclosed method produces about 70 mg of purified CPS from 4.8 liters of cell culture compared to previous methods which produce about 36 mg of purified CPS from the same amount of cell culture. In one embodiment, the disclosed method of isolating and purifying CPS is superior to previous methods because it does not involve the use of phenol, nucleases, proteases, and ultracentrifugation. In some embodiments, the disclosed method of isolating and purifying CPS is superior to previous methods because it can easily be scaled for use in industrial processes to isolate large quantities of Burkholderia CPS. Therefore, in some embodiments, the disclosed method further contemplates the use of steps similar to (a)-(h), as well as optional steps (i) and (j) with modifications such that they can be carried out at an industrial scale. In some embodiments, the disclosed method can be used to isolate and purify Burkholderia CPS from 50 mL or greater of cell culture on an industrial scale.

In another aspect, the present disclosure relates to a Burkholderia CPS that is isolated and purified using the disclosed method. In one embodiment, the Burkholderia CPS is isolated and purified from O-polysaccharide mutant strain B. thailandensis BT2683 using the disclosed method. In one embodiment, a 6-deoxy-heptan CPS is isolated and purified using the disclosed method.

### EXPERIMENTAL EXAMPLES

The invention is further described in detail by reference to the following experimental examples. These examples are provided for purposes of illustration only, and are not intended to be limiting unless so specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compounds of the present invention and practice the claimed methods. The following working examples therefore, specifically point out the preferred embodiments of the present invention, and are not to be construed as limiting in any way the remainder of the disclosure.

### Example 1: Purification of CPS from Burkholderia thailandensis BT2683

### Materials and Methods

### Source of Reagents and Consumables

M9 Minimal Salts, 5× (Difco 248510)
Tryptone (Bacto 211705)
0.45 μM Rapid-Flow Bottle Filter Unit, 75 mm (Nalgene 295-4545)
0.45 μm Syringe Filters, 33 mm (Millex HA SLHA033SB)
BupH PBS pH 7.2 (Thermo 28372)
Sephadex G-50, medium (Sigma G50150)

### Sample Preparation

The samples S1 and S2 of BT2683 (about 5 mg each) were each dissolved in 525 μL D₂O (99.96% D, Cambridge Isotope Laboratories) and transferred into a 5-mm NMR tube.

### NMR Spectroscopy

NMR data were acquired at 50° C. on a Bruker Avance III spectrometer (1H, 600.13 MHz) equipped with a cryoprobe. Quantitative ¹H NMR spectra were acquired with spectral width of 9615 Hz, 16384 complex data points, 4 transients and total recovery delay of 62 s between each transient. ¹H chemical shifts were referenced to the previously reported positions of B. thailandensis CPS signals. The data were processed and analyzed in Mestrenova (version 14.1.1-24571).

### Polysaccharide Quantitation

Relative molar per-cents of the polysaccharides A, B and C were determined based on integral intensities of all their overlapping and non-overlapping signals. Variables a, b and c were defined as theoretical integral intensities corresponding to one hydrogen in a repeat unit of polysaccharide A, B and C, respectively. Theoretical integral intensity of each signal was then expressed as a sum of coefficients a, b and c, each multiplied by number of hydrogens of A, B and C represented in the signal (Table 1). The values of coefficients a, b and c were determined by non-linear regression in Excel Solver by minimizing the sum of squared differences between the experimental and theoretical integral intensities.

TABLE 1
Integral ranges and expressions for theoretical integrals.

| Integral range (ppm) | Assignment | Theoretical integral |
|---|---|---|
| 5.36-5.19 | A2 + C2 | a + c |
| 5.14-5.05 | B1 | b |
| 4.95-4.81 | A1 + C1 | a + c |
| 4.24-4.15 | B2 | b |
| 4.03-3.85 | A3 + B3 + B6 + C3 | a + 2b + c |
| 3.85-3.67 | A7 + A7′ + B4 + B5 + B6′ | 2a + 3b |
| 3.58-3.33 | A4 + A5 + C5 + C4 | 2a + 2c |
| 2.21-2.04 | A6 + AAc + CAc | 4a + 3c |
| 1.81-1.63 | A6′ | a |
| 1.38-1.25 | C6 | 3c |

### Bt-CPS Purification Procedure

### Day 1

1. Inoculate 25 ml of M9 Minimal Salts+Tryptone (M9TG) in a 125 ml Erlenmeyer flask using a frozen stock of B. thailandensis BT2683 (O-polysaccharide mutant; ΔrmlD) and incubate ˜18 hrs @ 37° C. with vigorous shaking (˜200 rpm). This is the starter culture.

### Day 2

2. Inoculate 8×600 ml of M9TG in 2 L baffled flasks with 1 ml of culture (per flask) from the 125 ml Erlenmeyer flask and incubate 24 hrs @ 37° C. with vigorous shaking (˜200 rpm).

### Day 3

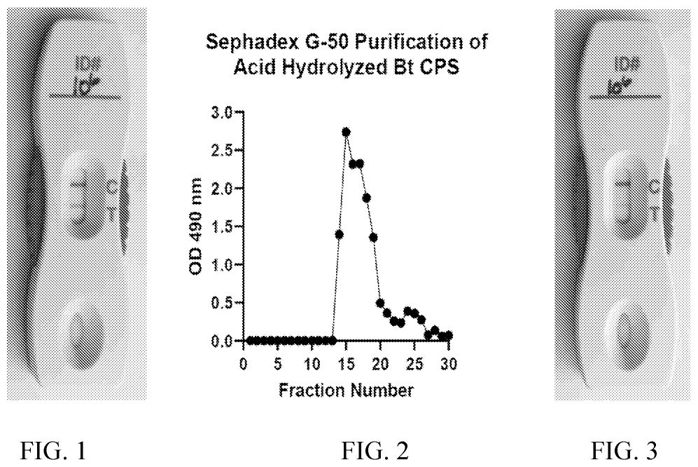

3. Pellet the cells (10 minutes @ 8,000×g) and resuspend in a total of 200 ml of dH₂O. Add the bacterial suspension to a 1 L Pyrex bottle containing a stir bar. Add an equal volume of absolute EtOH to the bottle and stir (˜400 rpm) at room temperature for 30 minutes.
4. Pellet the cell slurry (10 minutes @ 8,000×g) and carefully transfer the supernatant to a 1 L Pyrex bottle. Filter sterilize the supernatant into 2 L Pyrex bottle using a 0.45 μm Rapid-Flow Bottle Filter Unit.
5. Add 1600 ml of absolute EtOH to the supernatant to achieve a final concentration of 90% EtOH (v/v). Gently mix then let stand at room temperature (with periodic swirling) for 60 minutes. Pellet the insoluble material (30 minutes @ 12,000×g), carefully decant the supernatant and resuspend the CPS pellets in a total of 30 ml of dH₂O (FIG. 1). Dialyze the solubilized CPS against dH₂O using 3500 MWCO tubing. Periodically mix the contents in the dialysis tubing.

### Day 4

6. Clarify the dialysate with a 0.45 μm syringe filter then lyophilize to concentrate.

### Day 5

7. The mass of the lyophilized material was ˜120 mg. Solubilize the CPS @ 5 mg/ml in 2% acetic acid and incubate at @ 100° C. in a heating block for 2 hours. Periodically invert tube to mix. Use of a 50 ml Teflon tube at this stage is recommended.

8. Cool to room temperature and clarify the hydrolysate via centrifugation (20 minutes @ 17,000×g). Carefully remove the supernatant and lyophilize to concentrate.

### Day 6

9. The mass of the Bt-CPS lyophilized material was ˜96 mg. Solubilize the acid hydrolyzed sample @ ˜25 mg/ml in PBS pH 7.2 and clarify with a 0.45 μm syringe filter.

### Gel Filtration Procedure

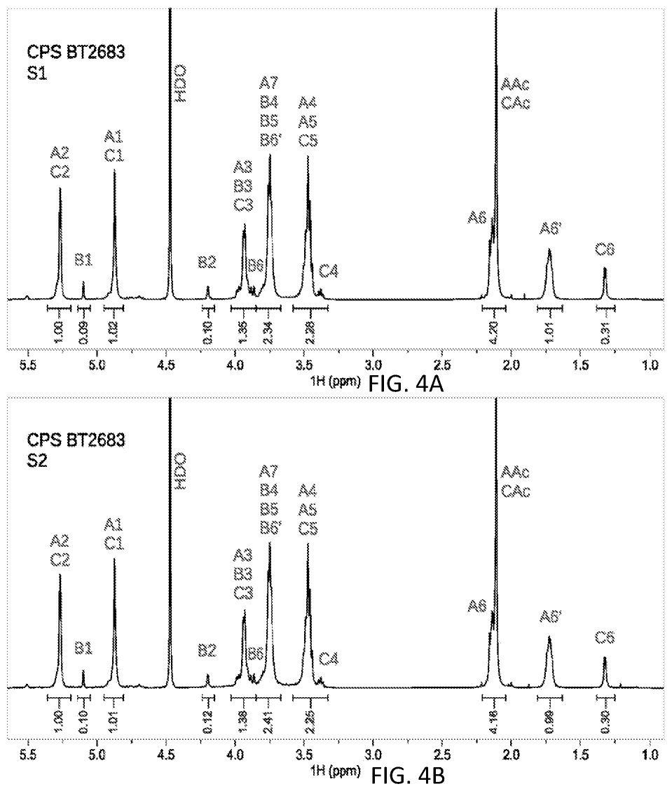

10. Load the Bt-CPS sample onto a Sephadex G-50 column (40 cm×2.6 cm; ˜180 ml resin) equilibrated with PBS pH 7.2 and eluted at a flow rate of about 0.75 ml/min with the same buffer. Assay fractions (˜6 ml) for carbohydrate using the phenol-sulfuric acid assay (FIG. 2). Note that alternatively the column can be equilibrated with water wherein the sample will then be eluted with water.
11. Pool fractions #13-19, dialyze against dH₂O using 3500 MWCO tubing then pass through a 0.45 μm syringe filter (FIG. 3). Note that this step is not necessary if water is used instead of PBS to prepare the Sephadex column and to elute the CPS.

### Day 8

12. Lyophilize the dialysate to concentrate.

### Day 9

The mass of the G-50 column purified BT2683 CPS was ˜70 mg.
Note: CPS can be eluted from the G-50 column using dH₂O as the buffer (solvent) system. Use of water as the solvent system can decrease the purification time from nine days to seven days.

### Results

The ¹H NMR spectra of BT2683 samples eluted from Sephadex G-50 columns in PBS (S1; FIG. 4A) or water (S2; FIG. 4B). were practically identical. The spectra were very similar to those of B. thailandensis CPS acquired previously and contained predominantly signals of a mixture of three different polysaccharides. Signal assignment of the three polysaccharides, shown in Table 2, was based on chemical shift comparison with previously assigned B. thailandensis CPS. Because only one isolated signal was available for polymers A and C, and in order to minimize bias in quantification due to potential overlaps of the isolated signals with underlying signals of minor species, relative quantification of the three polysaccharides was done by employing a fitting procedure which utilized all available polysaccharide signals (see Methods).

TABLE 2
Assignment of the ¹H NMR signals of BT2683 CPS.

| | | Atom chemical shift (ppm) | | | | | | | |
|---|---|---|---|---|---|---|---|---|---|
| Letter | Residue | 1 | 2 | 3 | 4 | 5 | 6 | 7 | OAa |
| A | →3-(2-OAc)-6d-β-manHep-1→ | 4.88 | 5.27 | 3.94 | 3.47 | 3.47 | 1.72 | 3.75 | 2.11 |
| | | | | | | | 2.14 | 3.75 | |
| B | →3-α-Man-1→ | 5.10 | 4.20 | 3.94 | 3.76 | 3.76 | 3.76 | | |
| | | | | | | | 3.88 | | |
| C | →3-(2-OAc)- β-Rha-1→ | 4.88 | 5.28 | 3.97 | 3.38 | 3.47 | 1.32 | | 2.11 |

The resulting polysaccharide composition of BT2863 samples S1 and S2 is shown in rTable 3. The amounts of polysaccharides A and B were slightly different in samples S1 and S2.

TABLE 3
The polysaccharide composition of BT2683 samples.

Relative mol %

| Letter | Residue | S1 | S2 |
|---|---|---|---|
| A | →3-(2-OAc)-6d-β-manHep-1→ | 81.2 | 79.8 |
| B | →3-a-Man-1→ | 10.3 | 12.1 |
| C | →3-(2-OAc)-β-Rha-1→ | 8.5 | 8.1 |

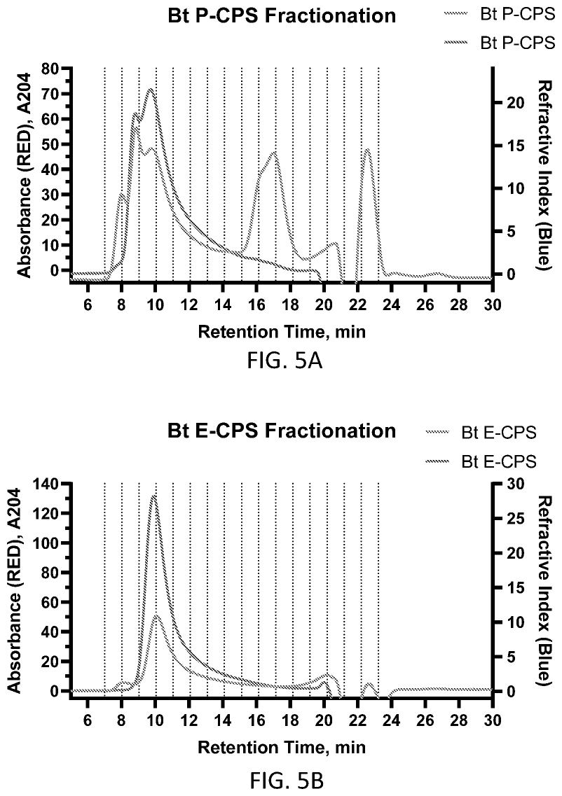

To compare the composition of Bt-CPS obtained using the classical phenol extraction method (P-CPS) or the ethanol extraction method (E-CPS), samples solubilized at 2 mg/ml in PBS were purified over G-50 resin and subsequently run on a Superdex 200 Increase 10/300 GL column. Results of the analyses (FIG. 5) show that the E-CPS material (FIG. 5B) is more uniform in size (i.e. less polydisperse) and contains less contaminants than the P-CPS material (FIG. 5A). These observations correlate with the higher quality glycoconjugate material (e.g. CPS-CRM197) that is produced using E-CPS material instead of P-CPS material.

The disclosed ethanol precipitation method is both faster and produces a higher yield and more uniform material than previous phenol extraction methods of purifying Burkholderia CPS. Further, the disclosed method takes only seven to nine days to arrive at purified CPS compared to twenty days for previous methods. The disclosed method also routinely produces about 70 mg of CPS from 4.8 liters of cell culture compared to phenol extraction methods which produce about 36 mg of CPS from the same amount of culture. Furthermore, the disclosed method is more readily scalable and more cost-effective than previous Burkholderia CPS purification methods because it does not use phenol, ultracentrifugation, or enzymes.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the disclosure. The appended claims are intended to be construed to include all such embodiments and equivalent variations.

### Enumerated Embodiments

Embodiment 1 provides a method for isolating and purifying a capsular polysaccharide (CPS) from a Burkholderia species, the method comprising (a) culturing Burkholderia cells; (b) pelleting the cells, resuspending the cells in water, adding an alcohol to the resuspended cells to form a cell slurry; (c) pelleting the cell slurry, removing the supernatant; (d) adding an alcohol to the supernatant, forming a precipitate comprising the CPS; (e) pelleting the CPS precipitate, resuspending the precipitate in water, dialyzing the resuspended CPS precipitate; (f) concentrating the dialyzed CPS precipitate, solubilizing the CPS in a weak acid, separating the solubilized CPS from a solid impurity; (g) concentrating the solubilized CPS, adding a buffer to the concentrated solubilized CPS to form a solution, loading the solution onto a gel filtration resin; and (h) eluting purified CPS from the gel filtration resin.

Embodiment 2 provides the method of embodiment 1, wherein the Burkholderia species is Burkholderia thailandensis.

Embodiment 3 provides the method of embodiment 1 or 2, wherein the Burkholderia species is an O-polysaccharide mutant strain of B. thailandensis.

Embodiment 4 provides the method of embodiment 3, wherein the O-polysaccharide mutant strain of B. thailandensis is B. thailandensis BT2683.

Embodiment 5 provides the method of any one of embodiments 1-4, wherein the CPS is a 6-deoxy-heptan CPS.

Embodiment 6 provides the method of any one of embodiments 1-5, wherein, step (b) comprises adding an equal volume of ethanol compared to the volume of resuspended cells, with stirring, to the resuspended cells to form a cell slurry.

Embodiment 7 provides the method of any one of embodiments 1-6, wherein step (d) comprises adding ethanol to the supernatant to achieve a final concentration of about 90% (v/v) ethanol, forming a precipitate comprising the CPS.

Embodiment 8 provides the method of any one of embodiments 1-7, wherein step (e) comprises dialyzing the resuspended CPS precipitate against water.

Embodiment 9 provides the method of any one of embodiments 1-8, wherein step (f) comprises solubilizing the CPS in an aqueous solution comprising between about 1% to 3% by volume of acetic acid to form a mixture having a concentration of between about 2 to 7 mg/ml CPS.

Embodiment 10 provides the method of embodiment 9, wherein the mixture is incubated for about 2 hours at a temperature of about 100° C.

Embodiment 11 provides the method of any one of embodiments 1-10, wherein step (g) comprises adding phosphate-buffered saline to the concentrated CPS to form a solution having a concentration of between about 20 to 30 mg/ml of the CPS and loading the solution onto a gel filtration resin, wherein the gel filtration resin comprises a cross-linked dextran gel filtration resin.

Embodiment 12 provides the method of any one of embodiments 1-10, wherein step (g) comprises adding deionized water to the concentrated CPS to form a solution having a concentration of between about 20 to 30 mg/ml of the CPS and loading the solution onto a gel filtration resin, wherein the gel filtration resin comprises a cross-linked dextran gel filtration resin.

Embodiment 13 provides the method of embodiment 11, wherein step (h) comprises eluting purified CPS from the gel filtration resin using phosphate-buffered saline.

Embodiment 14 provides the method of embodiment 12, wherein step (h) comprises eluting purified CPS from the gel filtration resin using deionized water.

Embodiment 15 provides the method of any one of embodiments 1-11, wherein step (h) is followed by step (i) comprising dialyzing the purified CPS.

Embodiment 16 provides the method of embodiment 15, wherein the purified CPS is dialyzed against water.

Embodiment 17 provides the method of any one of embodiments 1-16, wherein step (h) or step (i) is followed by step (j) comprising concentrating the purified CPS.

Embodiment 18 provides the method of any one of embodiments 1-16, wherein the alcohol is ethanol.

Embodiment 19 provides a Burkholderia CPS isolated and purified from O-polysaccharide mutant strain B. thailandensis BT2683 by the method of any one of embodiments 1-18.

Embodiment 20 provides a 6-deoxy-heptan CPS isolated and purified by the method of any one of embodiments 1-18.

1. A method for isolating and purifying a capsular polysaccharide (CPS) from a Burkholderia species, the method comprising
(a) culturing Burkholderia cells;
(b) pelleting the cells, resuspending the cells in water, adding an alcohol to the resuspended cells to form a cell slurry;
(c) pelleting the cell slurry, removing the supernatant;
(d) adding an alcohol to the supernatant, forming a precipitate comprising the CPS;
(e) pelleting the CPS precipitate, resuspending the precipitate in water, dialyzing the resuspended CPS precipitate;
(f) concentrating the dialyzed CPS precipitate, solubilizing the CPS in a weak acid, separating the solubilized CPS from a solid impurity;
(g) concentrating the solubilized CPS, adding a buffer to the concentrated CPS to form a solution, loading the solution onto a gel filtration resin; and
(h) eluting purified CPS from the gel filtration resin.

2. The method of claim 1, wherein the Burkholderia species is Burkholderia thailandensis.

3. The method of claim 1, wherein the Burkholderia species is an O-polysaccharide mutant strain of B. thailandensis.

4. The method of claim 3, wherein the O-polysaccharide mutant strain of B. thailandensis is B. thailandensis BT2683.

5. The method of claim 1, wherein the CPS is a 6-deoxy-heptan CPS.

6. The method of claim 1, wherein, step (b) comprises adding an equal volume of ethanol compared to the volume of resuspended cells, with stirring, to the resuspended cells to form a cell slurry.

7. The method of claim 1, wherein step (d) comprises adding ethanol to the supernatant to achieve a final concentration of about 90% (v/v) ethanol, forming a precipitate comprising the CPS.

8. The method of claim 1, wherein step (e) comprises dialyzing the resuspended CPS precipitate against water.

9. The method of claim 1, wherein step (f) comprises solubilizing the CPS in an aqueous solution comprising between about 1% to 3% by volume of acetic acid to form a mixture having a concentration of between about 2 to 7 mg/ml CPS.

10. The method of claim 9, wherein the mixture is incubated for about 2 hours at a temperature of about 100° C.

11. The method of claim 1, wherein step (g) comprises adding phosphate-buffered saline to the concentrated CPS to form a solution having a concentration of between about 20 to 30 mg/ml of the CPS and loading the solution onto a gel filtration resin, wherein the gel filtration resin comprises a cross-linked dextran gel filtration resin.

12. The method of claim 1, wherein step (g) comprises adding deionized water to the concentrated CPS to form a solution having a concentration of between about 20 to 30 mg/ml of the CPS and loading the solution onto a gel filtration resin, wherein the gel filtration resin comprises a cross-linked dextran gel filtration resin.

13. The method of claim 1, wherein step (h) comprises eluting purified CPS from the gel filtration resin using phosphate-buffered saline.

14. The method of claim 1, wherein step (h) comprises eluting purified CPS from the gel filtration resin using deionized water.

15. The method of claim 1, wherein step (h) is followed by step (i) comprising dialyzing the purified CPS.

16. The method of claim 15, wherein the purified CPS is dialyzed against water.

17. The method of claim 15, wherein step (h) or step (i) is followed by step (j) comprising concentrating the purified CPS.

18. The method of claim 1, wherein the alcohol is ethanol.

19. A Burkholderia CPS isolated and purified by the method of claim 1.

20. A Burkholderia CPS isolated and purified from O-polysaccharide mutant strain B. thailandensis BT2683 by the method of claim 1.

21. A 6-deoxy-heptan CPS isolated and purified by the method of claim 1.
